(19) United States
(12) Patent Application Publication
(10) Pub. No.: US 2013/0296740 A1
(43) Pub. Date: Nov. 7, 2013

(54) Method and Apparatus for Analyzing Foot Arch Deterioration

(71) Applicants: Kent S. Greenawalt, Roanoke, VA (US); Sean M. Naef, Roanoke, VA (US); Edward E. Quirouette, Vinton, VA (US)

(72) Inventors: Kent S. Greenawalt, Roanoke, VA (US); Sean M. Naef, Roanoke, VA (US); Edward E. Quirouette, Vinton, VA (US)

(21) Appl. No.: 13/465,366

(22) Filed: May 7, 2012

(51) Int. Cl. A61B 5/103

(52) U.S. Cl. 600/592

(57) ABSTRACT

A method and apparatus for helping corrective footwear professionals determine a need for corrective footwear comprises using an electronic scanner to determine a first area of the sole of the foot in contact with the scanning equipment within an area of interest and a second area of the sole of the foot not in contact with the scanning equipment within an area of interest. The percentage of the first area to the sum of the first and second areas is used to calculate a pronation/stability index. The pronation/stability index may be used by the corrective footwear professionals to determine the need for corrective footwear.

### BACKGROUND OF THE INVENTION

1. Field of the Invention

This invention relates generally to a method and apparatus for analyzing foot images to determine foot arch deterioration and a need for corrective footwear. More particularly, the present invention is connected with the use of a foot scanning hardware and software for obtaining and manipulating foot images and using those data to evaluate the integrity of the foot arches.

2. Background Art

In the making of orthopedic appliances such as custom orthotics, corrective footwear, and to aid in the correction or prevention of malformations of the feet, it is necessary to obtain information about the condition of the foot, such as deterioration of the arch. In the past, the measurements were taken using foam box casting—a method of taking the impressions of the feet in foam. A more convenient way to accomplish the task is by optical scanning of the feet.

Measurements may be made of scanned images of a subject's feet for the purpose of producing custom orthotics or footwear. This eliminates the potentially messy and time consuming task of taking impressions in foam. However, deterioration of the foot arch has not been a part of the results of a foot scan.

There is, therefore, a need for a method and apparatus for determining deterioration of the arches and quantifying the pronation and stability of the subject's foot based on a foot scan.

### SUMMARY OF THE INVENTION

It is an object of this invention to provide a novel method and apparatus for obtaining data associated with a patient's feet and calculating a measure of pronation and stability to aid in diagnosis and treatment of health problems related to arch deterioration.

A specialized scanner, with its associated software, captures an image of the bottom of a subject's foot in a weight-bearing position. The software is employed to analyze the foot image, looking for areas displaying various magnitudes of pressure. An algorithm is employed to colorize the foot according to the amount of pressure indicated. A color scheme is applied to the foot image, differentiating the zones of the foot based on the pressure applied thereto. The software then identifies key points of the foot based on the colorization. The software uses these points to dissect the foot into different zones. Each zone on the foot is tied to the corresponding part such as the forefoot, heel, toes, medial arch, transverse arch, and lateral arch. The software analyzes the color patterns in each of these zones to determine the integrity of the foot arches.

The algorithm is used to calculate the deterioration of the three foot arches and assign a pronation/stability index based on the sum of the calculation. The pronation/stability index is used to determine the need for custom orthotics, corrective shoes, or corrective sandals, and the construction thereof. For the purposes of this document, including the claims, the term corrective footwear shall be defined to include corrective orthotics, corrective shoes, or corrective sandals.

The pronation/stability index provides a corrective footwear professional a measure, the magnitude of which is indicative of the need for corrective footwear. The greater the pronation/stability index, the greater the need for correction. So the value of the calculated pronation/stability index may be shown to the subject and a range of values and their meanings relayed. A new scan may be taken at a later date and a new value of the pronation/stability index may be calculated to determine if the subject's arches have deteriorated, and if so, how much.

The novel features which are believed to be characteristic of this invention, both as to its organization and method operation together with further objectives and advantages thereto, will be better understood from the following description considered in connection with accompanying drawings in which a presently preferred embodiment of the invention is illustrated by way of example. It is to be expressly understood however, that the drawings are for the purpose of illustration and description only and not intended to define limits of the invention.

### BRIEF DESCRIPTION OF THE DRAWINGS

FIG. 1 shows a foot scanner with a subject standing thereon, and a display;

FIG. 2 shows a scan of a subject's feet;

FIG. 3 shows a line drawing of a foot of length, L;

FIG. 4 shows a colorized scan of a subject's feet divided up into subregions;

FIG. 5 shows a colorized scan of a subject's feet showing an Area of Interest;

FIG. 6 shows a subset of pixels used to display the colorized image of the feet; and

FIG. 7 shows a database used to organize and store subjects' data.

### BEST MODE FOR CARRYING OUT THE INVENTION

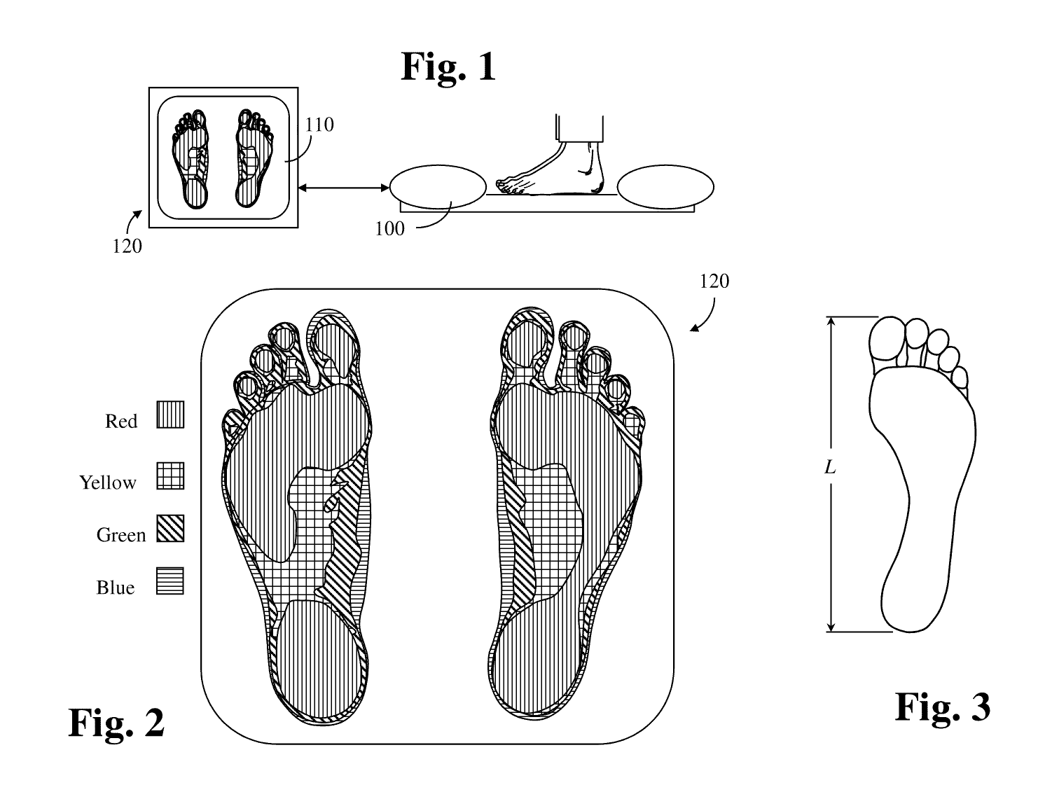

A scanner 100, depicted in FIG. 1 is used in the present invention to obtain electronic color images 120 of the bottom surfaces of a subject's feet. The subject stands on the scanner 100 in a natural weight bearing manner. The scanner is connected, electronically, to computing and displaying equipment 110. Image processing software running on the computing equipment 110 organizes the data to display them in a useful fashion to an operator.

Shown in FIG. 2 are colorized foot images 120 as displayed in the displaying equipment 110. The bottom surfaces of the feet are scanned with the scanner 100 and the foot images 120 colorized by the software running on the computing equipment, which may reside, physically, in the scanner or displaying equipment or another location.

The color scheme observed in FIG. 2 is meant to color the portion of the soles of the feet pressed against the scanner surface a first color, for instance red, as in FIG. 2. For the purposes of this document, including the claims, the color used to indicate the portion of the soles of the feet pressed against the scanner surface is hereby defined as the surface contact color. Regions of the foot not in contact with the scanner surface are colored at least a second color, not the same as the first color. As shown in FIG. 2, other colors, such as yellow, green, and blue, may be used for regions of the foot not in contact with the scanner surface. A color, for example, white, not used for any of the regions of the foot is used to indicate the area of the image that is not a part of the feet. For the purposes of this document, including the claims, this last region is hereby defined as the surroundings, and the color applied to the surroundings is defined as the surroundings color. The surroundings are areas in the image 120 that are not part of the image of the subject's feet.

Each foot has a length, L, as shown in FIG. 3. For the left foot, this length will be given the symbol, L_{L}, whereas the length of the right foot will be given the symbol, L_{R}. In general, L_{L} #L_{R}.

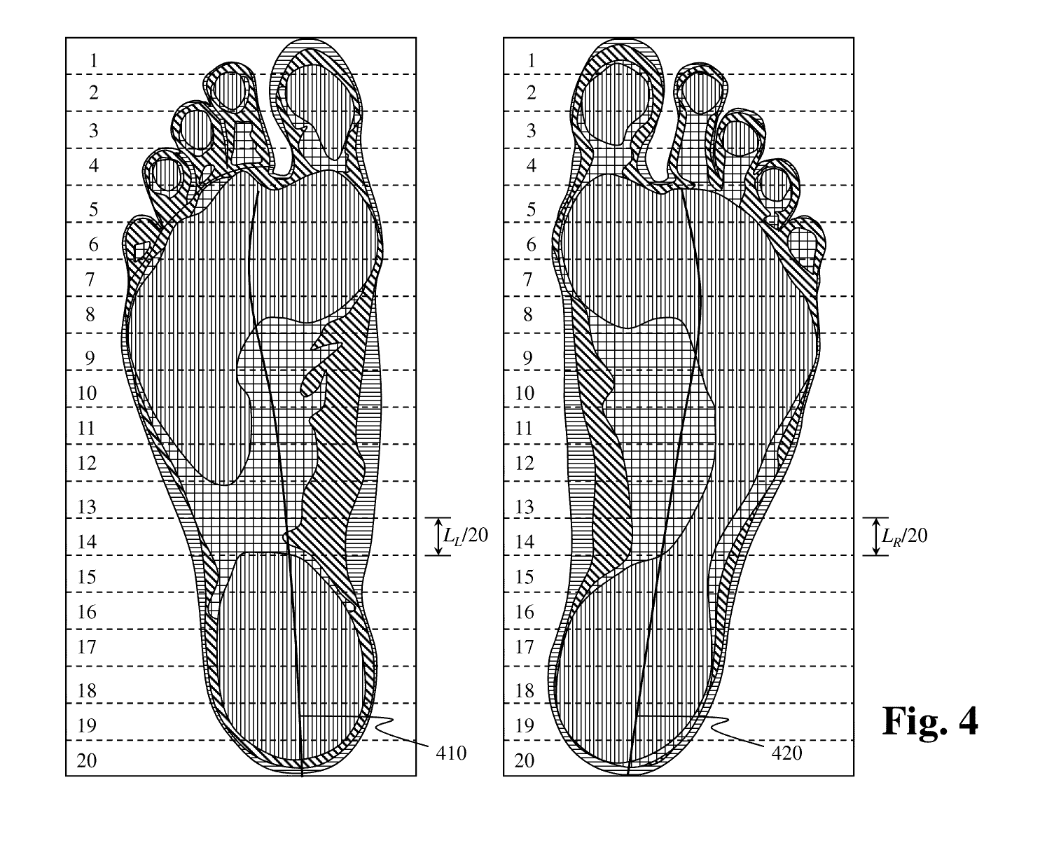

These lengths, L_{L} and L_{R}, are each divided by 20 in the preferred embodiment, so each foot image is divided into twenty areas, the height of all regions being equal, as shown in FIG. 4. The height of each region of the left foot is L_{L}/20, while for the right foot, the height of each region is L_{R}/20, as displayed in FIG. 4.

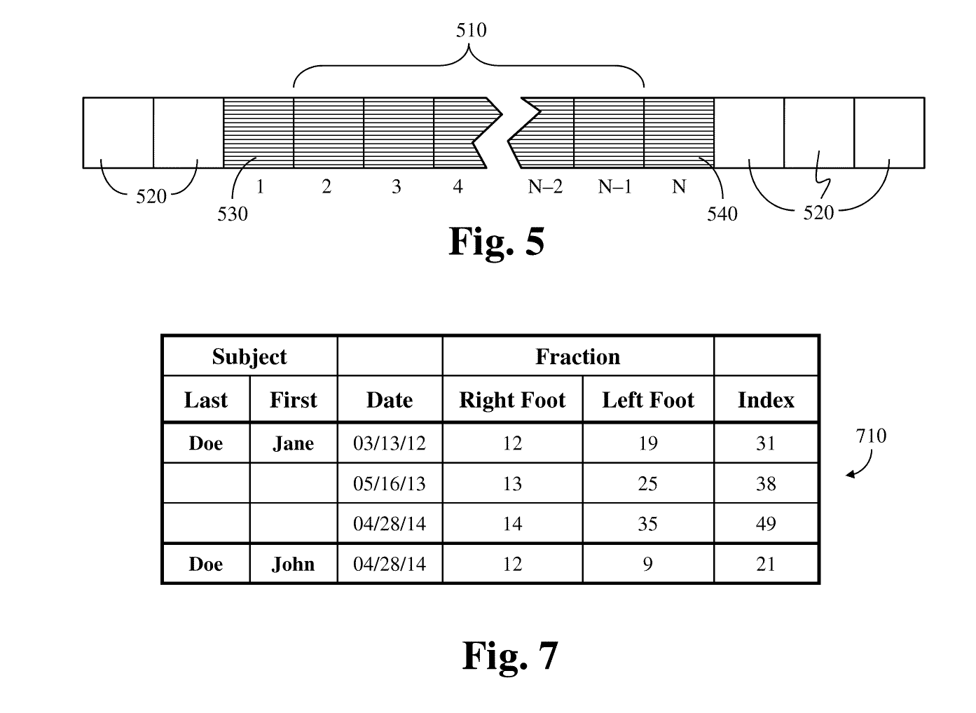

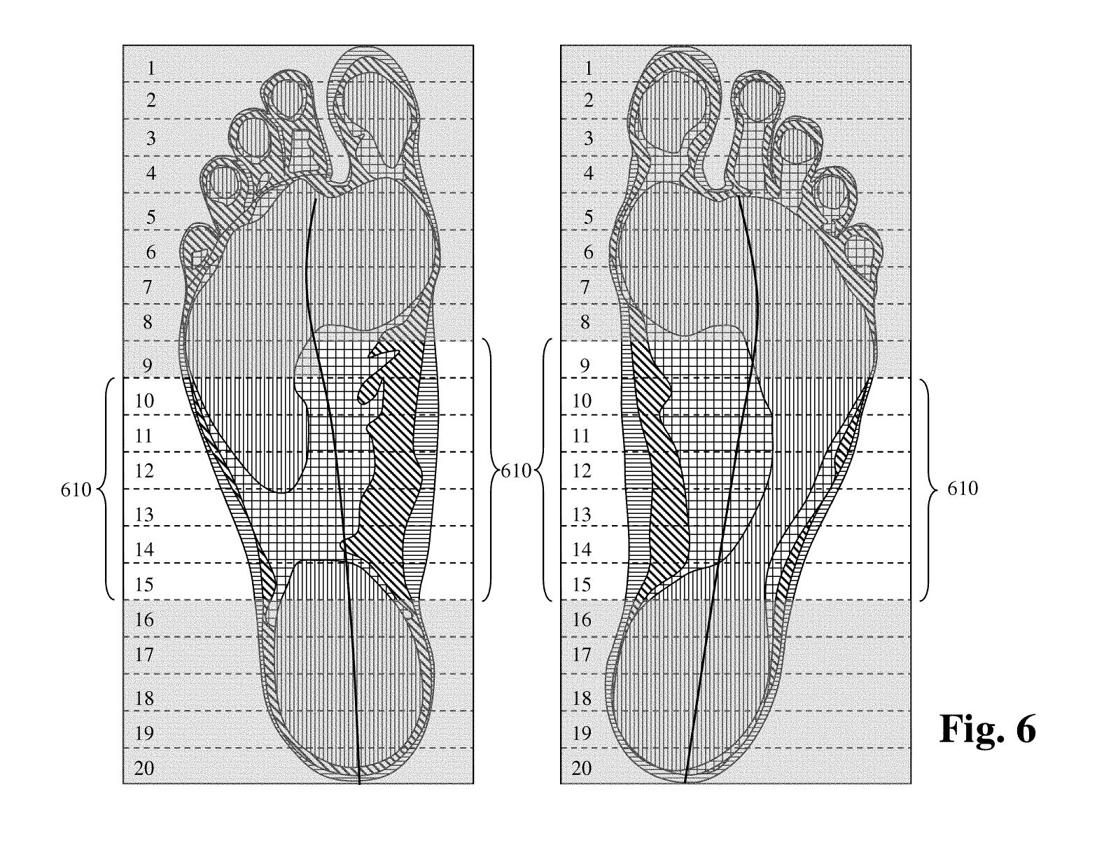

The pixels comprising the display are used to determine the midlines 410, 420 of the images of the soles of the feet. Representative pixels 510, 520, 530, 540 are shown in FIG. 5. The midline 410, 420 of each foot is determined by finding the midpoint between the leftmost pixel 530 and rightmost pixel 540 bordering the pixels 520 in the surroundings in each row of pixels. If white is used to depict the surroundings, as seen in FIG. 5, the midline of a foot would be located at the midpoint between the leftmost nonwhite pixel 530 and rightmost nonwhite pixel 540 in each row of pixels. If there are an odd number of nonwhite pixels 510, 530, 540 in a given row such that N/2 is not an integer, N/2 may be rounded up or down without loss of accuracy in the process. In the preferred embodiment, the midline of each foot is strictly required only for region number 9 (see FIG. 6) to define the Area of Interest 610.

The Area if Interest 610 for the each foot is defined as the seven half-areas (areas 9 through 15) on the inside of the foot, plus the six half-areas (areas 10 through 15) on the outside of the foot. The Area of Interest 610 region is shown in white in FIG. 6.

Within the Area of Interest 610, the number of pixels 510, 520, 530, 540 colored the surface contact color are numbered to determine a first value. The number of all pixels 510, 520, 530, 540 in the Area of Interest 610 colored any color other than the sourroundings color are also counted to determine a second value. A percentage of surface contact color to all non-surface color pixels 510, 520, 530, 540 is calculated by dividing the second value into the first value and multiplying by 100. This percentage is calculated for each foot, separately. The percentages found for the right foot and the left foot images are summed to produce a Pronation/Stability Index.

The Pronation/Stability Index provides information used by a corrective footwear professional. First, it indicates the level of need for corrective footwear. A higher value of the Pronation/Stability Index implies a greater need for corrective footwear. Second, the value may be recorded and the measurement and calulation repeated to learn of deterioration of the arches over time or the rate of deterioration. Whether or not corrective footwear is used by the subject, this historical record is useful to the footwear professional. To this end, the computing equipment may also include a database 710, as illustrated in FIG. 7, wherein subjects' records could be kept and updated for review and comparison.

The preferred embodiment of the present invention calls for the following analysis of the pronation/stability index:

Optimal: 0-34 Optimal. Feet have normal arches. Stabilizing orthotics can be worn for preventative care to avoid pain or injuries.

Mild: 35-84 Mild pronation. Some arch collapse has occurred. Stabilizing orthotics are recommended for people with mild pronation to avoid further arch collapse which can lead to pain throughout the body.

Moderate: 85-124 Moderate pronation. Nearly full arch collapse that could result in knee, hip, back or shoulder problems. It is necessary for moderate pronators to wear stabilizing orthotics to prevent even further damage.

Severe: ≧125 Severe pronation. Complete arch collapse and may have had knee, hip, back or shoulder problems due to this collapse. It is vital severe pronators wear stabilizing orthotics.

This invention may be practiced differently than described above. For example, it should be clear that the foot lengths, L_{L} and L_{R} may be divided up into a number of subregions other than 20; many combinations of pixel colors may be used; an average of the percentages of contact areas to all areas of the foot may be used instead of the sum to calculate the pronation/stability index; the pronation/stability index can be based on a fraction instead of a percentage—or any value proportional to the fraction; and the area of interest may be defined somewhat differently, all without materially changing the present invention.

The above embodiment is the preferred embodiment, but this invention is not limited thereto. It is, therefore, obvious that many modifications and variations of the present invention are possible in light of the above teachings. It is, therefore, to be understood that within the scope of the appended claims, the invention may be practiced otherwise than as specifically described.

1. A method of diagnosing a need for corrective footwear, the method comprising:
(a) scanning a sole of a foot with a scanner to produce a scanned image comprising the sole of the foot;
(b) determining an area of interest within the scanned image;
(c) determining a contact portion of the sole of the foot in contact with the scanner within the area of interest;
(d) determining a noncontact portion of the sole of the foot not in contact with the scanner within the area of interest;
(e) calculating a fraction by dividing the first portion by a sum of the first portion and the second portion; and
(f) determining a need for corrective footwear based on the fraction.

2. The method of claim 1 wherein the foot comprises a first foot of a subject and the fraction comprising a first fraction, the subject also having a second foot, the method further comprising:
(a) scanning a second sole of the second foot with a scanner to produce a second scanned image comprising the second sole of the foot;
(b) determining a second area of interest within the second scanned image;
(c) determining a second contact portion of the second sole of the second foot in contact with the scanner within the second area of interest;
(d) determining a second noncontact portion of the second sole of the second foot not in contact with the scanner within the second area of interest;
(e) calculating a second fraction by dividing the second contact portion by a sum of the second contact portion and the second noncontact portion;
(f) calculating a pronation/stability index proportional to a sum of the first fraction and the second fraction; and
(g) making a determination of a need for corrective footwear based on the pronation/stability index.

3. The method of claim 2 additionally comprising:
(a) determining a plurality of pronation/stability indices by scanning and calculating repeatedly over time; and
(b) determining a rate of deterioration of a subject's arches based on the plurality of pronation/stability indices.

4. The method of claim 1 wherein determining the area of interest within the scanned image comprises:
(a) determining a length of the foot;
(b) dividing the length of the foot into a plurality of sub-areas; and
(c) selecting a set of the plurality of sub-areas as the area of interest.

5. The method of claim 4 additionally comprising:
(a) detecting a left edge of the subject's foot based on a color of pixels in a row of a plurality of pixels within the scanned image; and
(b) detecting a right edge of the subject's foot based on the color of pixels in the row of the plurality of pixels within the scanned image.

6. The method of claim 5 additionally comprising:
(a) determining a midpoint of the subject's foot by calculating a number of pixels between the left edge of the subject's foot and the right edge of the subject's foot and dividing said number of pixels by two; and
(b) subdividing at least one of the plurality of sub-areas at the midpoint.

7. The method of claim 1 additionally comprising (a) assigning a first color to the contact portion of the sole of the foot in contact with the scanner; and
(b) assigning a second color to the noncontact portion of the sole of the foot not in contact with the scanner.

8. The method of claim 7 wherein determining the contact portion of the sole of the foot comprises counting a number of pixels in the area of interest of the scanned image having the first color.

9. The method of claim 7 wherein determining the noncontact portion of the sole of the foot comprises counting a number of pixels in the area of interest of the scanned image having the first color.

10. An apparatus for diagnosing a need for corrective footwear:
(a) at least one foot scanner, said foot scanner able to bear a weight applied to a foot of a human subject;
(b) computing equipment on which software can run;
(c) displaying equipment on which to view results of a scan and calculations;
(d) a computational algorithm to determine an area of interest within the scanned image, determine a contact portion of the sole of the foot in contact with the scanner within the area of interest, determine a noncontact portion of the sole of the foot not in contact with the scanner within the area of interest, and calculate a fraction by dividing the first portion by a sum of the first portion and the second portion; and
(e) hardcopy equipment to provide a printed results that come from the computational algorithm to the human subject.

11. The apparatus of claim 10 additionally comprising a database wherein subjects' data may be stored and organized.
